(19) United States
(12) Patent Application Publication
(10) Pub. No.: US 2013/0011437 A1
(43) Pub. Date: Jan. 10, 2013

(54) Immune compositions for treating H. pylori infection

(75) Inventor: Howard G. Fass, Danvers, MA (US)

(73) Assignee: LIFE SPRING IMMUNO SCIENCE CORP.

(21) Appl. No.: 13/135,387

(22) Filed: Jul. 5, 2011

(51) Int. Cl. A61K 39/02; A61P 31/04; A61P 37/04

(52) U.S. Cl. 424/234.1

(57) ABSTRACT

An immune composition for use, via topical administration, in eliciting Th2 or regulatory T cell-mediated immune responses specific to H. pylori. The immune composition, free of both liposaccharides and mammalian proteins, contains a whole-cell H. pylori lysate, and optionally, a vitamin D compound, halofuginone, or a mixture thereof as the sole adjuvant.

### BACKGROUND OF THE INVENTION

Helicobacter pylori is a gram-negative microaerophilic bacterium capable of causing chronic inflammation in the stomach. H. pylori infection leads to chronic gastritis, gastric/duodenal ulcer, and stomach cancer.

Antibiotics are commonly used in treating H. pylori infection. However, they are known to cause side effects in many people, including nausea, vomiting, dizziness, headache and others. In addition, an increasing number of H. pylori infected patients are found to harbor antibiotic-resistant bacterial stains, rendering antibiotic treatment ineffective in these patients.

Vaccination has been suggested as an optimal approach for preventing H. pylori infection and treating disorders associated with the infection. However, there is little success in developing vaccines that can prevent H. pylori infection, eradicate existing H. pylori colonization in the stomach, or reduce the severity of gastric diseases caused by H. pylori infection. There is a need for developing new anti-H. pylori vaccines with high therapeutic efficacy.

### SUMMARY OF THE INVENTION

In one aspect, the present invention features an immune composition for use in eliciting H. pylori-specific immune responses mediated by Th2 cells or regulatory T cells. Formulated for topical administration, this composition contains a whole-cell H. pylori lysate, which can be prepared from a H. pylori strain defective in lipopolysaccharide synthesis. It is free of both lipopolysaccharides and mammalian proteins. Preferably, the composition is also substantially free of nucleic acids, i.e., containing less than 20% (e.g., 10%, 5%, or lower) of nucleic acids by weight.

The immune composition of this invention is either adjuvant-free or contains, as the sole adjuvant, a vitamin D compound (e.g., vitamin D3 or calcipotriol), halofuginone, or a mixture thereof.

In another aspect, the present invention features a method for eliciting immune responses against H. pylori by administering topically to a subject in need thereof an effective amount of any of the above-described immune compositions.

Also within the scope of this invention is use of the immune composition in manufacturing a medicament for use in eliciting anti-H. pylori immune responses.

The details of one or more embodiments of the present invention are set forth in the description below. Other features or advantages of this invention will be apparent from the following drawings and detailed description of two examples, and also from the appended claims.

### BRIEF DESCRIPTION OF THE DRAWINGS

The drawing is first described.

FIG. 1 is a chart showing the level of H. pylori-specific antibodies in mice treated with PBS or a fraction containing outer membrane proteins from H. pylori cells.

### DETAILED DESCRIPTION OF THE INVENTION

Described herein is an immune composition containing a whole-cell lysate of H. pylori, the immune composition being free of mammalian proteins and endotoxins (liposaccharides). Upon topical administration, this composition elicited, unexpectedly, anti-H. pylori immune responses mediated predominantly by Th2 or regulatory T cells. As known in the art, such immune responses can ameliorate gastric diseases caused by H. pylori infection. See Lee et al., Infect Immun, 2007. 75(6): p. 2699-707. Thus, the immune composition of this invention is effective in treating H. pylori infection and alleivating symptoms caused by the infection. The term “treating” used herein refers to the application or administration of the immune composition of this invention to a subject, who suffers from H. pylori infection, a symptom of the infection, or is at risk for the infection, with the purpose to cure, heal, alleviate, relieve, alter, remedy, ameliorate, improve, or affect the infection, the symptoms of the infection, or the risk for the infection.

The whole-cell H. pylori lysate to be used in the immune composition of this invention can be prepared from any H. pylori strain, preferably from a mutant H. pylori strain that is defective in liposaccharide synthesis, e.g., carrying one or more defective WecA, Wzk, and WaaL genes. See Hug, I., et al., PLoS Pathog, 2010. 6(3): p. e1000819. To prepare the whole-cell lysate, a H. pylori strain can be cultured in a serum-free medium under suitable conditions for a suitable period. The H. pylori cells can then be harvested and lyzed via a conventional method, e.g., sonication or treatment by a detergent/surfactant such as SDS. The cell lysate thus formed is centrifuged or filtered through a suitable filter to remove large cell debris. Preferably, the resultant cell lysate is further subjected to salting-out or DNase and RNase digestion to remove nucleic acids. When a H. pylori stain capable of synthesizing liposaccharides is used to prepare the whole-cell lysate, the liposaccharides should be removed from the lysate by methods known in the art, e.g., LPS affinity resins, two-phase extraction, and ion exchange chromatography.

The whole-cell lysate described above can be mixed with a pharmaceutically acceptable carrier or excipient to form an immune composition in use for eliciting immune responses against H. pylori. The carrier in this immune composition must be “acceptable” in the sense that it is compatible with the active ingredient of the composition, and preferably, capable of stabilizing the active ingredient and not deleterious to the subject to be treated. Suitable carriers/excipients for use in preparing the immune composition include, but are not limited to, saline, dextrose, glycerol, ethanol, and combinations thereof. The vaccine may further contain minor amounts of auxiliary substances such as wetting or emulsifying agents, or pH buffering agents.

In addition to the whole-cell lysate, the just-described immune composition can further contain a vitamin D compound, halofuginone, or a mixture thereof as the sole adjuvant. The term “a vitamin D compound” refers to naturally-occurring vitamin D molecules (e.g., vitamins D₁-D₅) and analogues thereof, such as calcipotriol (also known as calcipotriene, an analogue of vitamin D₃).

The immune composition mentioned above can be formulated for topical administration (i.e., transcutaneous immunization) following methods known in the art. See, e.g., U.S. Pat. Nos. 7,037,499, 7,527,802, 7,473,247, and 7,722,595. Formulations suitable for topical administration include liquid and semi-liquid preparations that can be absorbed by the skin. Examples of liquid and semi-liquid preparations include, but are not limited to, topical solutions, liniments, lotions, creams, ointments or pastes, gels, and emugels.

Topical solutions are homogeneous mixtures prepared by dissolving a whole-cell lysate described above in a solvent. The solutions may contain one or more pharmaceutically acceptable carriers/expicients to buffer, stabilize, or preserve the protein antigens in the lysate. Solvents commonly used for preparation of topical solutions are ethanol, water, glycerol, propylene glycol, or any other vehicles known in the art. Optionally, L-menthol can be added to a topical solution.

Lotions, a preferred formulation for treating large body area, are typically liquid or semiliquid preparations in which solid particles, including an active agent, are present in a water or alcohol base. They are usually suspensions of solids, and preferably, contain a liquid oily emulsion of the oil-in-water type. The insoluble matter in a lotion should be finely divided such that it applies to the skin surface without friction. Lotions typically contain suspending agents to produce better dispersions as well as compounds useful for localizing and holding the antigens in the lysate in contact with the skin, e.g., methylcellulose, sodium carbozymethyl-cellulose, or the like.

Creams are viscous liquid or semisolid emulsions, either oil-in-water or water-in-oil, containing cream bases. The cream bases are water-washable and contain an oil phase, an emulsifier, and an aqueous phase. The oil phase, also called the “internal phase,” is generally comprised of petrolatum and a fatty alcohol such as cetyl or stearyl alcohol. The aqueous phase usually, although not necessary, exceeds the oil phase in volume, and contains a humectant. The emulsifier in a cream formulation can be a nonionic, anionic, cationic, or amphoteric surfactant. Exemplary surfactants include sorbitan esters or polyoxyethylene derivatives thereof (e.g., polyoxyethylene fatty acid esters) and carboxypolymethylene derivatives (e.g., carbopol).

Ointments are semisolid preparations that are typically based on petrolatum or other petroleum derivatives. The specific ointment base should possess a number of desirable features appreciable by those skilled in the art, e.g., emolliency or the like. As with other carriers or vehicles, an ointment based should be inert, stable, nonirritating, and nonsensitizing. There are four types of suitable ointment bases: oleaginous bases, emulsifiable bases, emulsion bases, and water-soluble bases. See Remington: The Science and Practice of Pharmacy, 19^{th} Ed., at pages 1399 and 1404. Oleaginous ointment bases include, for example, vegetable oils, fats obtained from animals, and semidolid hydrocarbons obtained from petroleum. Emusifiable ointment bases, also known as absorbent ointment bases, contain little or no water and include, for example, hydroxystearin sulfate, anhydrous lanolin, and hydrophilic petrolatum. Emulsion ointment bases are either water-in-oil emulsions or oil-in-water emulsions, and include, but are not limited to, cetyl alcohol, glyceryl monostearate, lanolin, and stearic acid. Preferred water-soluble ointment bases are prepared from polyethylene glycols of varying molecular weight.

Pastes are semisolid dosage forms in which an active agent is suspended in a suitable base. Depending on the nature of the base, pastes are divided between fatty pastes and those made from single-phase aqueous gels. The base in a fatty paste is generally petrolatum or hydrophilic petrolatum of the like. The pastes made from single-phase aqueous gels generally incorporate carboxymethylcellulose or the like as a base.

Gels or emugels include a commonly known gel forming agent, such as cellulose derivatives (e.g., methyl cellulose, hydroxyethyl cellulose, and carboxymethyl cellulose), vinyl polymers (e.g., polyvinyl alcohols and polyvinyl pyrrolidones), carboxypoly-methylene derivatives (e.g., carbopol), pectins and gums (e.g., gum arabic and tragacanth, alginate, carrageenate, agar, or gelatin). The gel or emugel formulations may further contain an auxiliary agent commonly known in the art, such as preservative, or stabilizer.

Any of the above-mentioned immune compositions, in topical formulation, can be applied to a subject in need of the treatment, e.g., a human subject who is suffering from or at risk for H. pylori infection, following traditional immunization procedures. In one example, the immune composition is applied directed onto a skin area of the subject. In another example, it is first absorbed into a patch, which is then placed onto a skin area of the subject. When an adjuvant-free immune composition is used, a subject can be treated either with this composition alone, or in combination with a vitamin D compound, halofuginone, or a mixture thereof. In the latter case, the subject is first administered topically with a formulation containing a vitamin D compound, halofuginone, or a mixture thereof, and, after a suitable period (e.g., 1 to 7 days), administered topically with the adjuvant-free composition.

Without further elaboration, it is believed that one skilled in the art can, based on the above description, utilize the present invention to its fullest extent. The following specific examples are, therefore, to be construed as merely illustrative, and not limitative of the remainder of the disclosure in any way whatsoever. All publications cited herein are incorporated by reference.

### EXAMPLE 1

### Induction of Anti-H. pylori Antibodies by Transcutaneous Immunization

A wild-type H. pylori strain was cultivated in brucella broth supplemented with 10% fetal bovine serum under microaerobic conditions for 24 h at 37° C. The H. pylori cells thus cultured were collected, washed with PBS, and then re-suspended in PBS. The bacterial cells were broken down by sonication on ice. A fraction containing outer membrane proteins was collected and used for immunization.

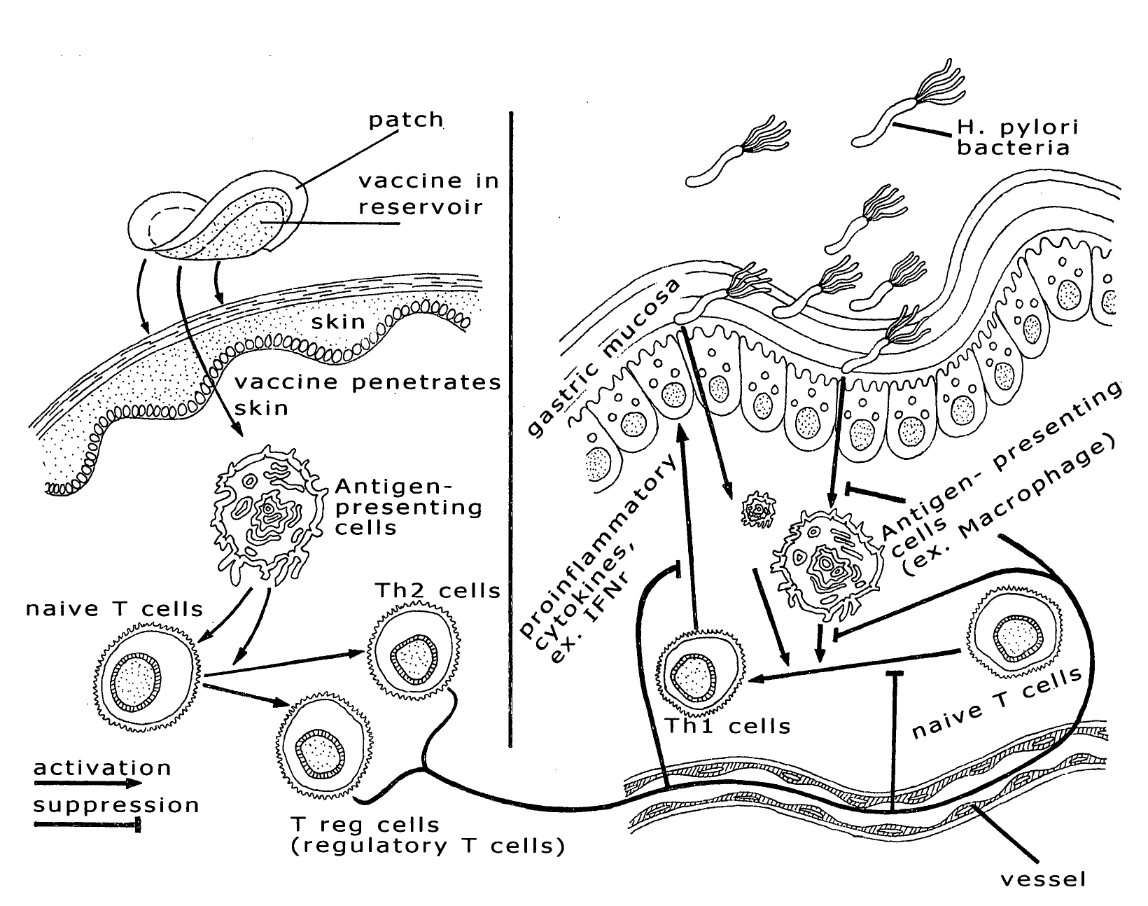

Mice were shaved and cleaned at certain skin areas and then immunized three times via transcutaneous delivery with (1) PBS, or (2) the H. pylori fraction that contains outer membrane proteins. Blood samples were collected from these mice 4 weeks post vaccination and the titers of anti-H. pylori antibodies in the sera were determined ELISA. As shown in FIG. 1, the titer of anti-H. pylori antibodies in the mice vaccinated with the membrane protein-containing fraction is significantly higher than that in the mice treated with PBS (P<0.05). This result indicates that transcutaneous immunization of a fraction containing H. pylori antigens (i.e., outer membrane proteins) successfully elicited antibody responses specific to H. pylori.

### EXAMPLE 2

### Preparation of Whole-Cell Lysate of H. pylori and Uses Thereof for Immunization Via Topical Administration

A mutated H. pylori strain defective in liposaccharide (endotoxin) synthesis is constructed by disrupting the function of WecA, Wzk, or WaaL gene, following the method described in Hug, I., et al., PLoS Pathog, 2010. 6(3): p. e1000819. This H. pylori strain is cultured in a Ham's F12-based, serum-free medium supplemented with cyclodextrins under microaerobic conditions. See Lee et al., Infect Immun, 2007. 75(6): p. 2699-707; and Testerman et al., J Clin Microbiol, 2001. 39(11): p. 3842-50.

The H. pylori cells thus cultured are collected, washed with PBS, and then re-suspended in PBS. The bacterial cells are broken down by sonication on ice. Large cell debris is removed by centrifugation or filtration through 0.22 μm filter to produce a whole-cell lysate. Nucleic acids are then removed from the cell lysate by precipitation with sodium chloride solution. Afterwards, the cell lysate is dialyzed against PBS or water. Absence of live bacteria in the cell lysate is confirmed via a conventional method, e.g., culturing.

The H. pylori cell lysate thus prepared is used as a vaccine composition for protecting against H. pylori infection. The following assay is performed to demonstrate its effectiveness in eliciting H. pylori-specific immune responses, particularly those mediated by Th1 or regulatory T cells, in mice.

Mice, 6-8 weeks old, are maintained in specific pathogen-free facilities. These mice are infected with H. pylori SS1 stain (see Lee et al., Gastroenterology, 1997. 112(4): p. 1386-97). Briefly, H. pylori cells are cultured under microaerobic conditions in brucella broth with 10% fetal bovine serum at 37° C. for 24 h, following the method described in Lee et al., Infect Immun, 2007. 75(6): p. 2699-707. The bacterial cells are harvested and suspended in PBS at OD₆₀₀ of 1.000, and then assessed by Gram staining and phase microscopy to determine purity, morphology, and motility, as well as activities of urease, catalase, and oxidase. Mice are administered with 0.2 ml of the bacterial suspension every other day for three doses. Control groups are given 0.2 ml of PBS. See Lee et al., Infect Immun, 2007. 75(6): p. 2699-707. The infected mice are sacrificed at 5 months post H. pylori infection.

The above-mentioned H. pylori-challenged mice are then administered with the H. pylori cell lysate described above, either four weeks before or four weeks after the H, pylori infection, as follows. More specifically, the cell lysate is applied to 1 cm² gauze on a patch, which is then placed on top of a shaved skin area (tape occluded) and secured with a bandage. See Wang et al., J Immunol, 1996. 156(11): p. 4077-82. For each course of immunization, the patch is left for 2 days and then replaced with another freshly prepared patch for another 2 days. Each mouse is immunized with the cell lysate every two weeks for two times.

Alternatively, mice are injected subcutaneously with the H. pylori cell lysate described above either one month before or one month after the H. pylori infection.

The immunized mice are sacrificed by CO₂ asphyxiation. Blood samples and gastric tissue samples are collected as described in Lee et al., Infect Immun, 2007. 75(6): p. 2699-707. Briefly, the stomach and proximal duodenum of each mouse are removed and the stomach is then incised along the greater curvature. Linear gastric strips from the lesser curvature are fixed overnight in 10% neutral-buffered formalin, embedded, cut to 4-μm thickness, and stained with H & E. Tissue sections are scored for gastric lesions by a veterinary pathologist.

The immunized mice are examined via ELISA and RT-PCR to determine levels of certain cytokines (i.e., IFN-γ, TNF-α, IL-2, IL-4, IL-10, IL-13, IL-17, and IL-23) in their gastric tissues and serum, respectively, following the method described in Lee et al., Cancer Res, 2008. 68(9): p. 3540-8. The type(s) of immune responses elicited by the H. pylori whole-cell lysate via topical administration can be determined based on the levels of the cytokines detected in the blood samples.

H. pylori colonization in the immunized mice is also analyzed by real-time quantitative PCR, following the method described in Lee et al., Cancer Res, 2008. 68(9): p. 3540-8.

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

What is claimed is:

1. An immune composition comprising a H. pylori whole-cell lysate, and optionally, a vitamin D compound, halofuginone, or a mixture thereof as the sole adjuvant, wherein the immune composition is formulated for topical administration and is free of both lipopolysaccharides and mammalian proteins.

2. The immune composition of claim 1, wherein the H. pylori whole-cell lysate is prepared from a H. pylori strain defective in lipopolysaccharide synthesis.

3. The immune composition of claim 1, wherein the H. pylori whole-cell lysate is substantially free of nucleic acids.

4. The immune composition of claim 1, wherein the immune composition is adjuvant-free.

5. The immune composition of claim 4, wherein the H. pylori whole-cell lysate is prepared from a H. pylori strain defective in lipopolysaccharide synthesis.

6. The immune composition of claim 4, wherein the H. pylori whole-cell lysate is substantially free of nucleic acids.

7. The immune composition of claim 1, wherein the composition contains a vitamin D compound, halofuginone, or a mixture thereof as the sole adjuvant.

8. The immune composition of claim 7, wherein the sole adjuvant is vitamin D3 or calcipotriol.

9. The immune composition of claim 7, wherein the sole adjuvant is halofuginone.

10. The immune composition of claim 7, wherein the sole adjuvant is a mixture of halofuginone and vitamin D3 or calcipotriol.

11. The immune composition of claim 7, wherein the H. pylori whole-cell lysate is prepared from a H. pylori strain defective in lipopolysaccharide synthesis.

12. The immune composition of claim 7, wherein the H. pylori whole-cell lysate is substantially free of nucleic acids.

13. A method for eliciting immune responses against H. pylori, the method comprising administering topically to a subject in need thereof an effective amount of an immune composition of claim 1.

14. The method of claim 13, wherein the H. pylori whole-cell lysate is prepared from a H. pylori strain defective in lipopolysaccharide synthesis.

15. The method of claim 13, wherein the H. pylori whole-cell lysate is substantially free of nucleic acids.

16. The method of claim 13, wherein the immune composition is adjuvant-free.

17. The method of claim 16, wherein, prior to administration of the adjuvant-free immune composition, the subject is further administered topically with a vitamin D compound, halofuginone, or a mixture thereof.

18. The method of claim 13, wherein the composition contains a vitamin D compound, halofuginone, or a mixture thereof as the sole adjuvant.

19. The method of claim 17, wherein the sole adjuvant is vitamin D3 or calcipotriol.

20. The method of claim 17, wherein the sole adjuvant is halofuginone.

21. The method of claim 17, wherein the sole adjuvant is a mixture of halofuginone and vitamin D3 or calcipotriol.
